SEQUENCE LISTING (6 sequences)

SEQ ID NO: 1 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 634 aa, synthetic construct
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAADILSLFCPDADKSCVASNNDQANINIESRSGRSYLPENRAVITPQGVTNWTYQELEATHQALTREGYVFVGYHGTNHVAAQTIVNRIAPVPRGNNTENEEKWGGLYVATHAEVAHGYARIKEGTGEYGLPTRAERDARGVMLRVYIPRASLERFYRTNTPLENAEEHITQVIGHSLPLRNEAFTGPESAGGEDETVIGWDMAIHAVAIPSTIPGNAYEELAIDEEAVAKEQSISTKPPYKERKDELK

SEQ ID NO: 3 - protein, 386 aa, synthetic construct
VEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQA

SEQ ID NO: 4 - protein, 561 aa, synthetic construct
MVEEALNIFDECRSPCSLTPEPGKPIQSKLSIPSDVVLDEGVLYYSMTINDEQNDIKDEDKGESIITIGEFATVRATRHYVNQDAPFGVIHLDITTENGTKTYSYNRKEGEFAINWLVPIGEDSPASIKISVDELDQQRNIIEVPKLYSIDLDNQTLEQWKTQGNVSFSVTRPEHNIAISWPSVSYKAAQKEGSRHKRWAHWHTGLALCWLVPMDAIYNYITQQNCTLGDNWFGGSYETVAGTPKVITVKQGIEQKPVEQRIHFSKGNAMSALAAHRVCGVPLETLARSRKPRDLTDDLSCAYQAQNIVSLFVATRILFSHLDSVFTLNLDEQEPEVAERLSDLRRINENNPGMVTQVLTVARQIYNDYVTHHPGLTPEQTSAGAQAGGGGSGGGGSGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 5 - protein, 14 aa, synthetic construct
GGGGSGGGGSGGGG

SEQ ID NO: 6 - DNA, 1683 bp, synthetic construct
  source 1..1683: mol_type = other DNA
atggtagaagaagcgctgaatatttttgacgagtgtcgtagcccgtgtagcctgactccggaaccgggcaagccgattcagtccaaactgtctatcccgagcgacgttgtgctggatgagggcgtgttgtattacagcatgacgatcaatgatgagcaaaacgacatcaaagacgaagataagggtgagtctattatcaccattggcgagtttgcgacggtgcgtgcaacgcgccattacgtgaatcaagacgcgccgtttggtgtcattcacctggatatcacgaccgaaaatggcacgaaaacctattcgtataatcgcaaagagggcgagttcgcgatcaactggctggttccgatcggtgaggatagcccggcgagcatcaagatcagcgttgatgaactggatcagcagcgcaacattattgaagtcccgaagttgtatagcatcgacctggataatcagaccctggagcagtggaaaacccagggtaacgttagcttctccgtgacccgtccggagcacaacattgccattagctggccgagcgttagctacaaagccgcacagaaagagggttcccgccacaagcgttgggctcattggcataccggtttggcgctgtgttggctggtgccgatggatgcgatctataactacatcacgcagcaaaattgcacgctgggtgacaattggttcggtggcagctacgagactgtggcgggtacccctaaggttattaccgtcaaacagggtattgagcaaaagcctgtcgagcagcgtatccactttagcaagggtaacgccatgtctgctctggcggctcatagagtttgcggcgttccgctggagactctggcccgttcccgcaagccgcgtgacctgaccgatgacctgagctgcgcgtatcaagcgcaaaacattgttagcttattcgttgcgacgcgcattttgttttcgcacctggatagcgtgttcacgctgaacctggatgaacaggaaccagaagtggcagagcgtctgtcagatctgcgtcgtatcaacgaaaacaacccgggcatggttacccaggtccttacggttgcacgccagatttacaatgattacgtgacccatcacccgggtctgaccccagaacaaaccagcgcaggcgcacaagcgggtggcggtggttccggtggcggtggtagcggtggcggtggtagccctggtcaaggcacccaatccgagaatagctgcacgcattttccaggcaatctgccgaatatgctgcgtgacctccgcgacgcgttctctcgtgttaagaccttttttcagatgaaagaccagctggacaatctgctgctgaaagaatccctgctggaagatttcaaaggctatctgggttgccaggccctgagcgagatgatccaattctacttggaagaggtcatgccgcaggccgaaaatcaagacccggacatcaaggcacacgtgaacagcttgggcgaaaacctgaaaaccctgcgtttgcgcctgcgtcgttgtcaccgtttcctgccgtgcgagaataagagcaaagccgtcgaacaagtcaaaaatgcattcaacaagctgcaagagaaaggtatctacaaggctatgagcgagtttgacattttcattaactacattgaagcgtacatgaccatgaagatccgtaac

(19) United States
(12) Patent Application Publication
(10) Pub. No.: US 2025/0235506 A1
(43) Pub. Date: Jul. 24, 2025

(54) CHIMERIC IL-10/CARRIER CONSTRUCTS FOR TREATMENT OF POUCHITIS AND METHODS OF USE

(71) Applicant: Thornhill Therapeutics, Inc., San Francisco, CA (US)

(72) Inventors: Bittoo Kanwar, San Francisco, CA (US); James Andrew Whitney, Mercer Island, WA (US); Kyler Lugo, Foster City, CA (US); Emily Weng, Foster City, CA (US); Drew Hotson, Redwood City, CA (US)

(21) Appl. No.: 18/906,129

(22) Filed: Oct. 3, 2024

Related U.S. Application Data
(63) Continuation of application No. PCT/US2023/017785, filed on Apr. 6, 2023.
(60) Provisional application No. 63/328,968, filed on Apr. 8, 2022.
(60) Provisional application No. 63/448,971, filed on Feb. 28, 2023.

(51) Int. Cl. A61K 38/20; A61K 9/00; A61K 9/20; A61K 38/45; A61P 29/00

(52) U.S. Cl. CPC A61K 38/2066; A61K 9/0053; A61K 9/2009; A61K 9/2013; A61K 9/2018; A61K 9/2027; A61K 9/2031; A61K 38/45; A61P 29/00; C12Y 204/02036

(57) ABSTRACT

Described herein are methods for treating pouchitis by orally administering a cholix-IL-fusion protein. Corresponding oral formulations and the effects resulting from administration of such oral formulations are disclosed.

### CROSS REFERENCE

This application is a continuation of International Application No. PCT/US2023/017785, filed Apr. 6, 2023, which claims the benefit of U.S. Provisional Application No. 63/328,968, filed Apr. 8, 2022, and U.S. Provisional Application No. 63/448,971, filed Feb. 28, 2023, each of which is incorporated herein by reference in entirety.

### SEQUENCE LISTING

The instant application contains a Sequence Listing which has been submitted electronically in XML format and is hereby incorporated by reference in its entirety. Said XML copy, created on Oct. 3, 2024, is named 67482-780.301.xml and is 9,695 bytes in size.

### BACKGROUND OF THE INVENTION

Approximately 30% of patients with ulcerative colitis (UC) eventually require total proctocolectomy within 15 years of diagnosis. Further, patients with severe UC that have failed an average of three lines of therapy can be considered for colectomy. Ileal pouch-anal anastomosis (IPAA) is currently the surgical treatment of choice as it avoids permanent ileostomy and is associated with better quality of life outcomes; IPAA can involve creation of a pouch of small intestine to recreate the rectum. Unfortunately, up to 50% of patients experience pouchitis within 10 years of IPAA, with most patients experiencing their first episode within 12 months, and some experience frequent recurrent episodes. Normal pouch function is typically 4 to 8 stools per day with 1 nocturnal motion and the ability to defer pouch evacuation. Pouchitis, on the other hand, is characterized by clinical symptoms of crampy lower abdominal pain, pelvic discomfort, general malaise, stool frequency above post-IPAA normal, urgency, incontinence, and nocturnal seepage. Some patients also present with extraintestinal symptoms in the joints, liver, eyes, or skin. The etiology of pouchitis is unknown, but it appears to involve multiple factors and is related to immune-mediated dysfunction in conjunction with an altered microbiotic environment. It is clear that pouchitis is more common in UC than other conditions that often lead to colectomy, such as familial adenomatous polyposis, suggesting that there are common pathogenetic mechanisms associated with UC and the development and progression of pouchitis.

The clinical symptoms of pouchitis can include erythema, mucous exudate, friability, bleeding, and/or ulcers of variable size. Histologic findings supportive of pouchitis can include acute inflammatory infiltrate with or without ulceration and crypt abscesses. Antibiotic therapy is usually successful in treating acute episodes, but approximately 10% to 20% of patients develop chronic pouchitis, including some patients that are responsive to antibiotics but require continuous therapy as well as patients who continue to experience symptoms/recurrence despite antibiotic treatment.

There are no approved therapies for pouchitis and treatment of chronic pouchitis is often challenging. While there is evidence that antibiotics can induce remission in some patients, there has been significant heterogeneity in response, and the benefits of long-term antibiotic use are unclear.

Studies have shown that mice that lack IL-10 expression spontaneously present signs of intestinal inflammation as early as 3 weeks of age. The symptoms progress into chronic transmural inflammation of the large and small bowels that resemble Crohn's disease (CD), which can be alleviated with IL-10 treatment. Furthermore, IL-10 has been identified in human genome-wide association studies as being associated with risk factors for IBD, adding further support for the contribution of IL-10 pathways to disease pathogenesis. Moreover, polymorphisms in the IL-10 receptor and loss-of-function mutations in the IL-10 gene or the IL-10 receptor are associated with early onset of disease for UC and CD, respectively. However, no IL-10 containing construct has been approved for use in treating human pouchitis, let alone a construct that can be delivered orally to the pouchitis patient.

While oral administration can be a convenient and desirable route for the administration of protein pharmaceuticals, challenges presented by this administration route include the acidic environment of the stomach, which can cause denaturation of protein structure (including disruption of the quaternary structure of an active protein complex, such as a homodimer), and hydrolysis of chemical bonds, variable pH across various regions of the gastrointestinal tract, and the presence of proteolytic enzymes which are secreted into the GI tract and break down proteins into smaller fragments. Furthermore, even if protein pharmaceuticals are able to survive these challenges and arrive intact in the lower GI tract, it can be difficult for such protein pharmaceuticals (such as IL-10) to cross the intestinal epithelium due to their large size. There is a need for improved methods and compositions for restoring intestinal immune homeostasis.

### SUMMARY OF THE INVENTION

In one aspect, provided herein is a method of treating an inflammatory disease of the gastrointestinal tract in a human subject to decrease stool frequency, the method comprising orally administering a therapeutically effective dose of a delivery construct consisting of SEQ ID NO: 4 to a human subject; wherein the oral administration of the therapeutically effective dose results in a decrease in stool frequency of the subject.

In some embodiments, the inflammatory disease of the intestinal tract is pouchitis. In some embodiments, the decrease in stool frequency is greater than or equal to 30% relative to stool frequency of the subject prior to administration of the delivery construct to the human subject. In some embodiments, the decrease in stool frequency is greater than or equal to three stools per day relative to stool frequency of the subject prior to administration of the delivery construct to the human subject. In some embodiments, the decrease in stool frequency results in a stool frequency that is at or below the stool frequency of the subject when the subject's bowel function was most settled after ileal pouch anal anastomosis. In some embodiments, the decrease in stool frequency results in a stool frequency that is at or below the stool frequency of the subject one year after ileal pouch anal anastomosis. In some embodiments, the decrease in stool frequency is measured by comparing stool frequency in week 12 to frequency prior to administration of the delivery construct to the human subject. In some embodiments, the decrease in stool frequency: (a) is (i) greater than or equal to three stools per day relative to stool frequency of the subject prior to administration of the delivery construct to the human subject and (ii) greater than or equal to 30% relative to stool frequency of the subject prior to administration of the delivery construct to the human subject; or (b) results in a stool frequency that is at or below the stool frequency of the subject when the subject's bowel function was most settled after ileal pouch anal anastomosis.

In some embodiments, the decrease in stool frequency: (a) is (i) greater than or equal to three stools per day relative to stool frequency of the subject prior to administration of the delivery construct to the human subject and (ii) greater than or equal to 30% relative to stool frequency of the subject prior to administration of the delivery construct to the human subject; or (b) results in a stool frequency that is at or below the stool frequency of the subject one year after ileal pouch anal anastomosis.

In some embodiments, the therapeutically effective dose of the delivery construct is 3 mg of the delivery construct. In some embodiments, the therapeutically effective dose of the delivery construct is 10 mg of the delivery construct. In some embodiments, the therapeutically effective dose is administered daily. In some embodiments, the therapeutically effective dose of the delivery construct is administered to the subject over a period of at least 12 weeks.

In some embodiments, the oral administration results in a histologic response. In some embodiments, the histologic response comprises neutrophil infiltration in less than 5% of the crypts. In some embodiments, the histologic response comprises no crypt destruction. In some embodiments, the histologic response comprises no erosions. In some embodiments, the histologic response comprises no ulcerations. In some embodiments, the histologic response comprises no granulation. In some embodiments, the histologic response results in a Geboes score of less than or equal to 3.1. In some embodiments, the histologic response is determined in week 12 after initial administration of the delivery construct to the human subject.

In some embodiments, the oral administration results in either (1) a decrease in pouchitis disease activity index (PDAI) histology subscore or (2) a PDAI histology subscore of 0. In some embodiments, the oral administration results in a decrease in PDAI subscore or PDAI score. In some embodiments, the oral administration results in a decrease in PDAI histology subscore of at least 2 points from a baseline pouchitis disease activity index histology subscore determined prior to administration of the delivery construct to the subject. In some embodiments, the decrease in pouchitis disease activity index histology subscore is determined at week 12 in comparison to a pouchitis disease activity index histology subscore determined prior to administration of the delivery construct to the human subject. In some embodiments, the oral administration results in a PDAI histology subscore of 0.

In some embodiments, the oral administration results in a greater than or equal to 50% reduction of simple endoscopic score for Crohn's disease (SES-CD) from baseline. In some embodiments, the reduction of SES-CD is measured by comparing an SES-CD score at week 12 to an SES-CD score prior to administration of the delivery construct to the human subject.

In some embodiments, the oral administration results in a PDAI neutrophil score ≤1 and ulcer score of 0.

In some embodiments, the oral administration results in increased Forkhead box P3 (FOXP3) positive T-regulatory cells in the Lamina propria. In some embodiments, the oral administration results in increased CD163+M2-macrophages in the Lamina propria. In some embodiments, the oral administration results in an increased phospho-STAT3 (Tyr705) to total STAT3 ratio in ileum. In some embodiments, the oral administration results in increased microbiome diversity in feces (e.g., as determined by an increase in a Shannon diversity score). In some embodiments, the oral administration results in decreased relative abundance of Proteobacteria [Pseudomonadota] in feces.

In some embodiments, the inflammatory disease of the gut (e.g., pouchitis) is chronic antibiotic-resistant pouchitis, chronic antibiotic-refractory pouchitis, or chronic antibiotic-dependent pouchitis.

In some embodiments, the subject has failed at least one round of antibiotic therapy prior to oral administration of the delivery construct to the human subject. In some embodiments, the subject does not receive an antibiotic during a course of treatment that comprises orally administering the therapeutically effective dose of the delivery construct consisting of SEQ ID NO: 4 to the human subject.

In some embodiments, the human subject had an mPDAI of greater than or equal to 5 prior to oral administration of the delivery construct to the human subject.

In some embodiments, the human subject had a Geboes score of greater than or equal to 3.1 prior to oral administration of the delivery construct to the human subject.

In some embodiments, the human subject had a daily stool frequency of greater than or equal to 6 prior to oral administration of the delivery construct to the human subject. In some embodiments, the human subject had an ileal pouch anal anastomosis (IPAA) at least one year before a first administration of the therapeutically effective dose of the delivery construct to the human subject, optionally wherein the human subject had three or more stools per day above a stool frequency of the human subject achieved post-IPAA when the human subject's bowel function was most settled. In some embodiments, the delivery construct is orally administered via a tablet.

In some embodiments, the tablet is an enteric-coated tablet. In some embodiments, the subject has been identified as having pouchitis prior to oral administration of the therapeutically effective dose.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates the structure of a cholix-IL-10 delivery construct homodimer (a dimer comprising two identical subunits of SEQ ID NO: 4) as determined by small angle X-ray scattering (SAXS).

FIG. 2 provides a table showing the determination of Geboes scores.

FIG. 3 provides a table showing the calculation of Pouchitis Disease Activity Index scores.

FIG. 4 provides a table showing the calculation of SES-CD values.

FIG. 5 provides a table showing St. Mark's Scale of Fecal Incontinence and Urgency.

FIG. 6 provides a table showing the baseline characteristics of patients in the Phase 2 clinical study.

FIG. 7 is a waterfall plot showing the percent change from baseline for FOXP3+ cells seen in biopsies from each patient treated with 3 mg or 10 mg doses of SEQ ID NO: 4.

FIG. 8 is a waterfall plot showing the percent change from baseline for CD163+ cells seen in biopsies from each patient treated with 3 mg or 10 mg doses of SEQ ID NO: 4.

FIG. 9 is a waterfall plot showing the percent change from baseline in the phospho-STAT3:total STAT3 ratio observed in colonic biopsies from each patient treated with 3 mg or 10 mg doses of SEQ ID NO: 4.

FIG. 10 provides a spaghetti plot of fecal microbiome alpha diversity measurements of each patient treated with 3 mg or 10 mg doses of SEQ ID NO: 4 both before treatment (at randomization) and after 12 weeks of treatment.

FIG. 11 provides a spaghetti plot of relative abundance of Proteobacteria in each patient treated with 3 mg or 10 mg doses of SEQ ID NO: 4 both before treatment (at randomization) and after 12 weeks of treatment.

### DETAILED DESCRIPTION OF THE DISCLOSURE

IL-10 is an anti-inflammatory cytokine which can limit the damage to tissues caused by infections or inflammation, making IL-10 an attractive protein for therapeutic drug development. A fusion protein comprising IL-10 and a carrier, referred to herein as an IL-10 delivery construct, can be formulated into a form suitable for oral administration, such as a tablet for treatment of a human having pouchitis and for restoring intestinal immune homeostasis. Additionally, enteric coatings around these oral formulations can contribute to a distinct dissolution profile of the IL-10 delivery construct.

IL-10 is considered a master regulator of the innate and adaptive immune system, as it is thought to inhibit not only the inflammasome but also many inflammatory events found to be associated with disease including macrophage activation and secretion of IL-1, IL-6, TNF alpha, MMP-1/2 while reducing systemic signs of inflammation and inducing the activation of T regulatory cells.

The below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise. It is further noted that the claims can be drafted to exclude any optional element. As such, this statement is intended to serve as antecedent basis for use of such exclusive terminology as “solely,” “only,” and the like in connection with the recitation of claim elements, or use of a “negative” limitation.

Certain ranges or numbers are presented herein with numerical values being preceded by the term “about.” The term “about” is used herein to mean plus or minus 1%, 2%, 3%, 4%, or 5% of the number that the term refers to. As used herein, the terms “subject” and “individual,” are used interchangeably and can be any animal, including mammals (e.g., a human or non-human animal).

As used herein, the terms “treat,” “treating” or “treatment,” and other grammatical equivalents, include alleviating, abating or ameliorating one or more symptoms of a disease or condition; ameliorating or preventing the underlying causes of one or more symptoms of a disease or condition; inhibiting the disease or condition, such as, for example, arresting the development of the disease or condition, relieving the disease or condition, causing regression of the disease or condition, relieving a condition caused by the disease or condition, or inhibiting the symptoms of the disease or condition either prophylactically and/or therapeutically.

As described herein, the term “percent (%) sequence identity” and terms related thereto, in the context of amino acid sequences or nucleic acid sequences, is the percentage of amino acid residues or nucleic acid residues in a candidate sequence that are identical with the amino acid residues or nucleic acid residues, respectively, in a selected sequence, after aligning the sequences and introducing gaps, if necessary, to achieve the maximum percent sequence identity, and not considering any conservative substitutions as part of the sequence identity. Alignment for purposes of determining percent amino acid sequence identity or percent nucleic acid identity can be achieved in various ways that are within the skill in the art, for instance, using publicly available computer software such as Clustal Omega, BLAST, BLAST-2, ALIGN, ALIGN-2 or Megalign (DNASTAR) software, with BLAST being the alignment algorithm of preference. Those skilled in the art can determine appropriate parameters for measuring alignment, including any algorithms needed to achieve maximal alignment over the full-length of the sequences being compared, although for simplicity it maybe preferred to use default parameters.

### Interleukin-10 (IL-10) and IL-10 Delivery Constructs

The present disclosure contemplates compositions and methods for delivery of IL-10 to a human subject having pouchitis (e.g., an individual who has been identified as having pouchitis). Human IL-10 exists in solution primarily as a homodimer, where two subunits of IL-10 are non-covalently associated and each subunit contains two intrachain disulfide bonds. Disruption of the dimer structure can cause the subunits to dissociate to produce monomers of IL-10 or aggregates thereof, which can lack the biological activity of the dimer. Biological activity associated with IL-10 in a dimer form can comprise induction of pro-inflammatory cytokines, such as, tumor necrosis factor alpha (TNFα), interleukin-1β (IL-1β), interleukin-12 (IL-12), and interleukin-6 (IL-6). Biological activity associated with IL-10 in a dimer form can comprise downregulation of the expression of Th1 cytokines, MHC class II antigens, and co-stimulatory molecules on macrophages; enhancing B cell survival, proliferation, and antibody production; blocking of NF-κB activity; and regulating the JAK-STAT pathway.

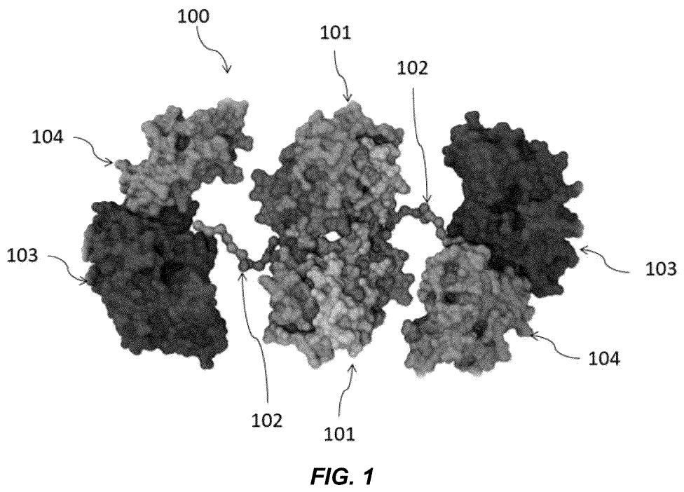

In the present disclosure, an IL-10 molecule is coupled to a carrier that can deliver the IL-10 across a gut epithelial cell, or a polarized epithelial cell. This is referred to as an IL-10 delivery construct. Preferably, the IL-10 that is coupled to the carrier is in a dimer form. An IL-10 delivery construct dimer can be illustrated by FIG. 1. The IL-10 delivery construct homodimer 100 can comprise two IL-10 delivery constructs (e.g., SEQ ID NO: 4), each delivery construct comprising an IL-10 101 connected by a spacer 102 to a carrier. The carrier can comprise a binding domain 103 and a translocation domain 104.

IL-10 can be a human IL-10. Human IL-10 can comprise, consist essentially of, or consist of the amino acid sequence of SEQ ID NO: 1. Variants of IL-10 include those having one or more amino acid substitutions, additions, and/or deletions as compared to a reference sequence. Variants of IL-10 may retain the ability to upregulate IL-1Ra in colonic tissue or serum after administration by intracolonic spray in cynomolgus monkeys. In some instances, variants of IL-10 or SEQ ID NO: 1 are contemplated in the compositions and methods described herein. Variants of IL-10 or SEQ ID NO: 1 can be an amino acid sequence having at least 80%, 85%, 90%, 95%, 98% or 99%% sequence identity thereto or a fragment thereof.

A carrier can be a protein or another type of molecule capable of transporting the heterologous payload across or into an epithelium (e.g., a polarized gut epithelium of a subject, such as a human). Such transport can include transcytosis. The transcytosis process may involve interaction(s) of the carrier with one or more receptor(s) and/or protein(s) on the apical and/or basal surface(s) as well as inside a cell of the epithelium (e.g., a polarized gut epithelial cell). The carrier can be capable of transporting a heterologous payload, such IL-10, across an epithelium without impairing the epithelium, the carrier, and/or the biological and/or therapeutic function of the payload.

In some embodiments, a carrier herein utilizes an endogenous trafficking pathway to transport a heterologous payload coupled thereto across a polarized epithelial cell. Such carrier can be referred to herein as a transcytosing carrier.

The carriers herein can transport molecules coupled thereto by interacting and/or co-localizing with one or more endogenous proteins of such epithelium. The one or more endogenous proteins can be receptors or enzymes capable of moving a carrier across the epithelial cell. Interacting and/or co-localizing with the one or more endogenous proteins of the epithelial cell can provide a carrier with one or more functions, including endocytosis into the epithelial cell, avoidance of a lysosomal destruction pathway, trafficking from an apical compartment to a basal compartment, and/or exocytosis from the basal membrane of the epithelial cell into a submucosal compartment such as the Lamina propria.

A carrier may be derived from a polypeptide secreted by a bacterium. Such a carrier may be derived from a polypeptide secreted from Vibrio cholerae or Pseudomonas aeruginosa. In some embodiments, the carrier is a cholix polypeptide. In some embodiments, the carrier is a cholix polypeptide secreted by Vibrio cholerae. A cholix polypeptide can include naturally and non-naturally occurring cholix polypeptide sequences, as well as those sequences that have at least about 75%, 80%, 85%, 90%, 95%, 98%, 99%, or 100% sequence identity to a portion of a naturally occurring or non-naturally occurring (e.g., SEQ ID NO: 2) cholix polypeptide described herein. A cholix polypeptide can also include a transcytosing fragment (e.g., N- and/or C-terminal truncations of cholix polypeptide) of naturally or non-naturally occurring cholix polypeptide sequences, wherein such endocytosing and/or transcytosing fragments can have at least about 75%, 80%, 85%, 90%, 95%, 98%, 99%, or 100% sequence identity to any of such naturally or non-naturally occurring cholix polypeptide sequences.

For example, a non-naturally occurring cholix polypeptide can include or consist of the amino acid sequence set forth in SEQ ID NO: 2 (TABLE 1). A cholix polypeptide carrier can be a truncated and/or mutated variant of a full-length cholix polypeptide. Mutation(s) in a non-naturally occurring variant can include one or more substitution(s), deletion(s), and/or addition(s) relative to a naturally occurring cholix polypeptide. In some embodiments, a carrier herein can comprise a V1L substitution. Stated differently, in some embodiments, the cholix-related carrier has a leucine amino acid at position “1.” (Position 1 generally refers to the first amino acid of variants that do not have an N-terminal methionine or the second position in variants that include an N-terminal methionine. In other words, in determining the length of a carrier, an N-terminal methionine, if present, can be ignored.) In some embodiments, carriers comprising the V1L substitution experience reduced or eliminated cleavage of the N-terminal amino acid. In some embodiments, carriers comprising the V1L substitution experience reduced or eliminated acetylation of the N-terminal amino acid. A carrier provided herein can have a reduced (e.g., at least 50% reduced) or ablated ADP ribosylation activity (e.g., ribosylation of elongation factor 2) relative to a naturally-occurring cholix variant. In some embodiments, the carrier can comprise an N-terminal methionine. In other embodiments, no N-terminal methionine is present.

A carrier herein can have a reduced (e.g., at least 50% reduced) or ablated ADP ribosylation activity (e.g., ribosylation of elongation factor 2). In another instance, a carrier comprises SEQ ID NO: 3. Any of the carriers herein can have a V1L substitution, alone or in combination with an N-terminal methionine. Carriers also include variants of any of the above having at least 80%, 85%, 90%, 95%, 98%, or 99% sequence identity to SEQ ID NO: 3.

A carrier can be coupled to the IL-10 covalently. When an IL-10 is coupled to a carrier covalently, it may be coupled to the carrier directly or via a spacer. The IL-10 can be coupled to the C-terminus or the N-terminus of the carrier. When a spacer is used to couple the IL-10 to the carrier, a spacer can include one or more amino acids. An example of an oligopeptide spacer sequence is SEQ ID NO: 5 (GGGGSGGGGSGGGG).

The carrier and/or the IL-10 can further comprise one or more modifications on their N-terminus and/or C-terminus. Such modifications can include an N-terminal methionine residue or other known residue for an expression in a heterologous system.

In some embodiments, the IL-10 delivery construct comprises, consists essentially of, or consists of the amino acid sequence of SEQ ID NO: 4. The IL-10 delivery construct can have at least 80%, 85%, 90%, 92%, 94%, 95%, 96%, 97%, 98%, 99%, or 100% sequence identity to an amino acid sequence set forth in SEQ ID NO: 4.

Further described herein are non-naturally occurring nucleic acids comprising, consisting essentially of, or consisting of a nucleic acid sequence set forth in SEQ ID NO:6 (encoding the delivery construct of SEQ ID NO: 4), or a nucleic acid sequence having at least 90%, at least 92%, at least 95%, at least 98%, or at least 99% sequence identity to SEQ ID NO: 6. The nucleic acid can be codon optimized. The nucleic acid can be encoded by a vector. The vector can be a plasmid or a viral vector. The viral vector can be a lentivirus, an adenovirus, an adeno-associated virus (AAV), a retrovirus, or a herpes simplex virus. The vector can be replication competent or a replication incompetent. The vector can be an integrating vector or a non-integrating vector. A cell can be transformed with any of the vectors described herein. The cell can be a bacterial cell. The bacterial cell can be an Escherichia coli cell.

TABLE 1
Sequences

| SEQ ID NO: | Description | Sequence |
|---|---|---|
| SEQ ID NO: 1 | IL-10, | SPGQGTQSENSCTHFPGNLPNMLRDLRDAFSRVKTFFQMKD |
| | secreted | QLDNLLLKESLLEDFKGYLGCQALSEMIQFYLEEVMPQAEN |
| | active form | QDPDIKAHVNSLGENLKTLRLRLRRCHRFLPCENKSKAVEQ |
| | | VKNAFNKLQEKGIYKAMSEFDIFINYIEAYMTMKIRN |
| SEQ ID NO: 2 | Non- | VEEALNIFDECRSPCSLTPEPGKPIQSKLSIPSDVVLDEGV |
| | naturally | LYYSMTINDEQNDIKDEDKGESIITIGEFATVRATRHYVNQ |
| | occurring | DAPFGVIHLDITTENGTKTYSYNRKEGEFAINWLVPIGEDS |
| | cholix | PASIKISVDELDQQRNIIEVPKLYSIDLDNQTLEQWKTQGN |
| | variant | VSFSVTRPEHNIAISWPSVSYKAAQKEGSRHKRWAHWHTGL |
| | | ALCWLVPMDAIYNYITQQNCTLGDNWFGGSYETVAGTPKVI |
| | | GTVKQIEQKPVEQRIHFSKGNAMSALAAHRVCGVPLETLAR |
| | | SRKPRDLTDDLSCAYQAQNIVSLFVATRILFSHLDSVFTLN |
| | | LDEQEPEVAERLSDLRRINENNPGMVTQVLTVARQIYNDYV |
| | | THHPGLTPEQTSAGAQAADILSLFCPDADKSCVASNNDQAN |
| | | INIESRSGRSYLPENRAVITPQGVTNWTYQELEATHQALTR |
| | | EGYVFVGYHGTNHVAAQTIVNRIAPVPRGNNTENEEKWGGL |
| | | YVATHAEVAHGYARIKEGTGEYGLPTRAERDARGVMLRVYI |
| | | PRASLERFYRTNTPLENAEEHITQVIGHSLPLRNEAFTGPE |
| | | SAGGEDETVIGWDMAIHAVAIPSTIPGNAYEELAIDEEAVA |
| | | KEQSISTKPPYKERKDELK |
| SEQ ID NO: 3 | Truncated | VEEALNIFDECRSPCSLTPEPGKPIQSKLSIPSDVVLDEGV |
| | non- | LYYSMTINDEQNDIKDEDKGESIITIGEFATVRATRHYVNQ |
| | naturally | DAPFGVIHLDITTENGTKTYSYNRKEGEFAINWLVPIGEDS |
| | occurring | PASIKISVDELDQQRNIIEVPKLYSIDLDNQTLEQWKTQGN |
| | cholix | VSFSVTRPEHNIAISWPSVSYKAAQKEGSRHKRWAHWHTGL |
| | variant | ALCWLVPMDAIYNYITQQNCTLGDNWFGGSYETVAGTPKVI |
| | (cholix386) | TVKQGIEQKPVEQRIHFSKGNAMSALAAHRVCGVPLETLAR |
| | | SRKPRDLTDDLSCAYQAQNIVSLFVATRILFSHLDSVFTLN |
| | | LDEQEPEVAERLSDLRRINENNPGMVTQVLTVARQIYNDYV |
| | | THHPGLTPEQTSAGAQA |
| SEQ ID NO: 4 | IL-10 | MVEEALNIFDECRSPCSLTPEPGKPIQSKLSIPSDVVLDEG |
| | delivery | VLYYSMTINDEQNDIKDEDKGESIITIGEFATVRATRHYVN |
| | construct | QDAPFGVIHLDITTENGTKTYSYNRKEGEFAINWLVPIGED |
| | | SPASIKISVDELDQQRNIIEVPKLYSIDLDNQTLEQWKTQG |
| | | NVSFSVTRPEHNIAISWPSVSYKAAQKEGSRHKRWAHWHTG |
| | | LALCWLVPMDAIYNYITQQNCTLGDNWFGGSYETVAGTPKV |
| | | ITVKQGIEQKPVEQRIHFSKGNAMSALAAHRVCGVPLETLA |
| | | RSRKPRDLTDDLSCAYQAQNIVSLFVATRILFSHLDSVFTL |
| | | NLDEQEPEVAERLSDLRRINENNPGMVTQVLTVARQIYNDY |
| | | VTHHPGLTPEQTSAGAQAGGGGSGGGGSGGGGSPGQGTQSE |
| | | NSCTHFPGNLPNMLRDLRDAFSRVKTFFQMKDQLDNLLLKE |
| | | SLLEDFKGYLGCQALSEMIQFYLEEVMPQAENQDPDIKAHV |
| | | NSLGENLKTLRLRLRRCHRFLPCENKSKAVEQVKNAFNKLQ |
| | | EKGIYKAMSEFDIFINYIEAYMTMKIRN |
| SEQ ID NO: 5 | Spacer | GGGGSGGGGSGGGG |
| SEQ ID NO: 6 | DNA | ATGGTAGAAGAAGCGCTGAATATTTTTGACGAGTGTCGTAG |
| | sequence | CCCGTGTAGCCTGACTCCGGAACCGGGCAAGCCGATTCAGT |
| | encoding | CCAAACTGTCTATCCCGAGCGACGTTGTGCTGGATGAGGGC |
| | IL-10 | GTGTTGTATTACAGCATGACGATCAATGATGAGCAAAACGA |
| | delivery | CATCAAAGACGAAGATAAGGGTGAGTCTATTATCACCATTG |
| | construct | GCGAGTTTGCGACGGTGCGTGCAACGCGCCATTACGTGAAT |
| | | CAAGACGCGCCGTTTGGTGTCATTCACCTGGATATCACGAC |
| | | CGAAAATGGCACGAAAACCTATTCGTATAATCGCAAAGAGG |
| | | GCGAGTTCGCGATCAACTGGCTGGTTCCGATCGGTGAGGAT |
| | | AGCCCGGCGAGCATCAAGATCAGCGTTGATGAACTGGATCA |
| | | GCAGCGCAACATTATTGAAGTCCCGAAGTTGTATAGCATCG |
| | | ACCTGGATAATCAGACCCTGGAGCAGTGGAAAACCCAGGGT |
| | | AACGTTAGCTTCTCCGTGACCCGTCCGGAGCACAACATTGC |
| | | CATTAGCTGGCCGAGCGTTAGCTACAAAGCCGCACAGAAAG |
| | | AGGGTTCCCGCCACAAGCGTTGGGCTCATTGGCATACCGGT |
| | | TTGGCGCTGTGTTGGCTGGTGCCGATGGATGCGATCTATAA |
| | | CTACATCACGCAGCAAAATTGCACGCTGGGTGACAATTGGT |
| | | TCGGTGGCAGCTACGAGACTGTGGCGGGTACCCCTAAGGTT |
| | | ATTACCGTCAAACAGGGTATTGAGCAAAAGCCTGTCGAGCA |
| | | GCGTATCCACTTTAGCAAGGGTAACGCCATGTCTGCTCTGG |
| | | CGGCTCATAGAGTTTGCGGCGTTCCGCTGGAGACTCTGGCC |
| | | CGTTCCCGCAAGCCGCGTGACCTGACCGATGACCTGAGCTG |
| | | CGCGTATCAAGCGCAAAACATTGTTAGCTTATTCGTTGCGA |
| | | CGCGCATTTTGTTTTCGCACCTGGATAGCGTGTTCACGCTG |
| | | AACCTGGATGAACAGGAACCAGAAGTGGCAGAGCGTCTGTC |
| | | AGATCTGCGTCGTATCAACGAAAACAACCCGGGCATGGTTA |
| | | CCCAGGTCCTTACGGTTGCACGCCAGATTTACAATGATTAC |
| | | GTGACCCATCACCCGGGTCTGACCCCAGAACAAACCAGCGC |
| | | AGGCGCACAAGCGGGTGGCGGTGGTTCCGGTGGCGGTGGTA |
| | | GCGGTGGCGGTGGTAGCCCTGGTCAAGGCACCCAATCCGAG |
| | | AATAGCTGCACGCATTTTCCAGGCAATCTGCCGAATATGCT |
| | | GCGTGACCTCCGCGACGCGTTCTCTCGTGTTAAGACCTTTT |
| | | TTCAGATGAAAGACCAGCTGGACAATCTGCTGCTGAAAGAA |
| | | TCCCTGCTGGAAGATTTCAAAGGCTATCTGGGTTGCCAGGC |
| | | CCTGAGCGAGATGATCCAATTCTACTTGGAAGAGGTCATGC |
| | | CGCAGGCCGAAAATCAAGACCCGGACATCAAGGCACACGTG |
| | | AACAGCTTGGGCGAAAACCTGAAAACCCTGCGTTTGCGCCT |
| | | GCGTCGTTGTCACCGTTTCCTGCCGTGCGAGAATAAGAGCA |
| | | AAGCCGTCGAACAAGTCAAAAATGCATTCAACAAGCTGCAA |
| | | GAGAAAGGTATCTACAAGGCTATGAGCGAGTTTGACATTTT |
| | | CATTAACTACATTGAAGCGTACATGACCATGAAGATCCGTA |
| | | AC |

### Oral Formulations

In some embodiments, the delivery construct (e.g., SEQ ID NO: 4) may be delivered orally to a subject (e.g., to treat pouchitis). In some embodiments, the delivery construct is delivered to the subject via a tablet or other oral formulation. In some embodiments, greater than 80%, greater than 85%, or greater than 90% of the IL-10 in the tablet is in a dimer form.

A formulation comprising IL-10 can be delivered to the small intestines or colon in a formulation described herein. The formulation can be delivered orally. In some embodiments, such formulations may facilitate crossing of the construct across the intestinal epithelial cell barrier (e.g., via transcytosis), which can otherwise prevent achievement of the full therapeutic potential of the IL-10. Furthermore, targeted delivery of IL-10 directly to gastrointestinal tissue via the oral route may bypass the side effects experienced with systemic administration and can translate into higher mucosal concentrations and clinically meaningful reductions in inflammation and disease.

In some embodiments, the oral formulation comprises an enteric coating (e.g., an enteric-coated tablet) for release in the gastrointestinal tract. In some embodiments, the oral formulation is configured for site site-specific release of the delivery construct in the terminal ileum, proximal colon, or distal colon.

The oral formulation for delivery of a therapeutic protein to the lower GI tract can comprise a tablet with a coating configured to dissolve at a pH found in the small intestines or colon, which has a pH in the range of from about 5.5 to about 8.0. In some embodiments, the coating is configured not to dissolve in the highly acidic pH of the stomach, which can range from a pH of about 1.5 to about 3.5.

An oral formulation herein can be configured to pass through the stomach without releasing the delivery construct to an appreciable extent. Release of the delivery construct can occur after full or partial dissolution of at least one coating on a tablet comprising the delivery construct.

The oral formulation can be in a unit dose form. In some instances, the oral formulation is provided in a therapeutically effective unit dose of 3 mg of delivery construct. In some instances, the oral formulation is provided in a therapeutically effective unit dose of 10 mg of delivery construct.

In some embodiments, the oral formulation comprises one or more excipients. In some embodiments, the one or more excipients can comprise a salt/buffer (e.g., potassium phosphate), glycine, sucrose, and/or a surfactant (e.g., poloxamer 188). In some embodiments, the one or more excipients can comprise an osmolyte. Osmolytes can be used in pharmaceutical formulations comprising proteins to improve stability of the proteins and decrease protein aggregation. The osmolyte can be an amino acid (e.g. glycine) and/or a sugar (e.g., sucrose).

In some embodiments, the one or more excipients can include a surfactant. Surfactants can be used in solid oral formulations comprising proteins, such as a tablet, to enhance disintegration of the solid oral formulation and increase solubility of the proteins. The surfactant can be poloxamer 188. The oral formulation can comprise a weight ratio of the surfactant to therapeutic protein from about 0.10:1 to about 0.19:1, from about 0.12:1 to about 0.18:1, from about 0.14:1 to about 0.16:1, from about 0.15:1 to about 0.17:1, from about 0.16:1 to about 0.17:1, about 0.15:1, or about 0.17:1. Use of a poloxamer as an excipient in the compositions described herein can promote or maintain dimerization of the IL-10 or IL-10 delivery construct.

The one or more excipients can include a salt. The salt can be potassium phosphate, sodium chloride or a combination thereof. The oral formulation can have a weight ratio of the salt to therapeutic protein from about 0.03:1 to about 0.15:1, from about 0.05:1 to about 0.12:1, from about 0.05:1 to about 0.09:1, from about 0.08:1 to about 0.12:1, or more preferably about 0.09:1 or about 0.10:1.

In some embodiments, the one or more excipients can include a bulking agent. Bulking agents can be used to increase the size of an oral formulation for ease of manufacturing. The bulking agent can be sucrose or glycine or a combination thereof. The oral formulation can comprise a weight ratio of glycine to therapeutic protein from about 0.7:1 to about 1.3:1, from about 0.8:1 to about 1.2:1, from about 0.9:1 to about 1.1:1, or about 1:1. The oral formulation can comprise a weight ratio of sucrose to therapeutic protein from about 0.3:1 to about 0.7:1, from about 0.4:1 to about 0.6:1, from about 0.45:1 to 0.55:1, or more preferably about 0.5:1. The oral formulation can comprise a weight ratio of bulking agent (e.g., glycine+sucrose) to therapeutic protein from about 1.2:1 to about 1.8:1, from about 1.3:1 to about 1.7:1, or from about 1.4:1 to about 1.6:1, or more preferably about 1.5:1.

The oral formulation or portions thereof (e.g., the drug substance) can be compressed under a compression force to produce a tablet. The compression force can range from 1500 pound-force (lbf) to 4000 lbf or from 2000 lbf to 3500 lbf. The compression force can be 1500 lbf, 2000 lbf, 2500 lbf, 3000 lbf, 3500 lbf, or 4000 lbf. The tablet can have a weight from 150 mg to 1000 mg, from 150 mg to 500 mg, from 200 mg to 400 mg, from 150 mg to 250 mg, from 175 mg to 225 mg, or from 190 mg to 210 mg. The tablet can comprise a diameter from 0.2″ to 0.4″, from 0.25″ to 0.35″, or from 0.3″ to 0.25″. The tablet can comprise 3 mg or 10 mg of the delivery construct. The tablet can be round, oblong, oval, circular, or any other suitable shape.

When the oral formulation is a tablet, the one or more excipients can include a compacting excipient. The one or more excipients can comprise 1, 2, 3, 4, or more than 4 compacting excipients. The compacting excipient can be a disintegrant, a lubricant, or a combination thereof. The IL-10 delivery construct can comprise from about 5% to about 15% (w/w) of the tablet.

The compacting excipients can comprise a disintegrant. A disintegrant can facilitate the dispersion or break up of an oral formulation. The disintegrant can comprise silicified microcrystalline cellulose (SMCC), crospovidone (crosslinked polyvinylpyrrolidone), or a combination thereof.

The compacting excipients can comprise a lubricant. A lubricant can reduce interparticle friction and cohesion in an oral formulation. The lubricant can comprise a non-ionic surfactant, such as glyceryl dibehenate.

The oral formulation can comprise a first coat disposed around an interior portion that contains the delivery construct. The first coat may comprise hydroxypropyl methylcellulose (HPMC). In some embodiments, the total weight of the first coat is approximately 8 mg. A second coat may be disposed exterior of the first coat. The second coat may function as an enteric coating that protects the interior contents of the oral formulation from contact with the stomach when administered orally. The second coat may be a Eudragit-containing coating. In some instances, the second coat has a 30:70 ratio (w/w) of Eudragit L30D55 to Eudragit FS30D. In some embodiments, the second coat further comprises a plasticizer, such as PlasAcryl T20 (between 9% and 11% PlasAcryl T20 on a w/w basis (solids content)). In some embodiments, the oral formulation is a tablet comprising a delivery-construct containing core and one or more coatings (e.g., a first HPMC coat and a second (exterior) Eudragit/plasticizer coating).

### Treatment

Described herein, in certain embodiments, are methods of treating a disease or condition in an individual in need thereof. The individual can be a human subject. The individual can be an individual diagnosed with or suspected of having pouchitis. Treating pouchitis can comprise administering a therapeutically effective amount of an IL-10 delivery construct to an individual suffering from (or suspected of suffering from) pouchitis.

The term, “therapeutically effective amount,” as used herein, can mean that the amount of an IL-10 delivery construct, contained in a composition, e.g. formulation or oral formulation described herein, administered is of sufficient quantity to achieve the intended purpose, such as, for example, to treat pouchitis or a symptom thereof. In some embodiments, administering a formulation to an individual comprises administering a therapeutically effective amount of the formulation to the individual.

The formulations comprising the IL-10 delivery construct can be administered to the individual for at least 1 day, at least 1 week, at least 2 weeks, at least 5 weeks, at least 10 weeks, or at least 12 weeks. The formulation can be administered once a day. The individual in need thereof can be a human. An individual in need thereof can be an individual diagnosed with, suspected of having an inflammatory disease of the gut, such as pouchitis. In some instances, the individual has been diagnosed with or is suspected of having chronic antibiotic-resistant pouchitis, chronic antibiotic-refractory pouchitis, or chronic antibiotic-dependent pouchitis. In some instances, the individual has failed at least one round of antibiotic therapy. In some instances, the individual does not receive an antibiotic during the course of treatment. In some instances, the individual had an mPADI of greater than or equal to 5. In some embodiments, the individual had a Geboes score of greater than or equal to 3.1. In some embodiments, the individual had 3 more stools per day above Normal (after IPAA). In some embodiments, the individual had a daily stool frequency of greater than or equal to 6. In some embodiments, the individual had an ileal pouch anal anastomosis at least one year before administration of a therapeutically effective does of a delivery construct described herein. The oral formulation can be used to treat the disorder causing inflammation. The method can comprise administering a dose of an oral formulation comprising the IL-10 delivery construct and one or more pharmaceutically acceptable excipients to an individual.

A response to administration of the IL-10 delivery construct can comprise a histologic response (e.g., a decrease in a Geboes score). A response to administration of the IL-10 delivery construct can comprise a decrease in a Geboes score relative to a Geboes score baseline. Geboes scoring system is a standard measure of histological response (Geboes et al. Gut. 2000 September; 47(3):404-9). A response to administration of the IL-10 delivery construct can comprise a decrease in stool frequency relative to a baseline stool frequency. A response to administration of the IL-10 delivery construct can comprise a decrease in Pouchitis Disease Activity Index (PDAI) score or subscore relative to a baseline. A response to administration of the IL-10 delivery construct can comprise a reduction in a simple endoscopic score for Crohn's disease (SES-CD).

Colonic tissue of an individual with pouchitis can show infiltration of the Lamina propria by mononuclear cells, eosinophils, and histiocytes, or a combination thereof in addition to neutrophilic infiltration into the epithelium associated with crypt architecture destruction, erosions, and ulcerations. Administration of an IL-10 delivery construct to an individual can results in a reduction in the infiltration of the Lamina propria by mononuclear cells, eosinophils, and histiocytes, or a combination thereof in addition to neutrophilic infiltration into the epithelium associated with crypt architecture destruction, erosions, and ulcerations. A response to administration of the IL-10 delivery construct can comprise a change in immune cell population in mucosal tissue. A response to administration of the IL-10 delivery construct can comprise a change in gene expression in mucosal tissue. A response to administration of the IL-10 delivery construct can comprise a change in fecal microbiome population and diversity.

### EXAMPLES

### Example 1: IL-10 Delivery Construct Design

IL-10 is an immunomodulatory cytokine that suppresses the activation and effector function of multiple innate and adaptive immune cells. An IL-10 delivery construct (SEQ ID NO: 4) was designed. This construct was a recombinant, homodimeric fusion protein where each monomer consisted of an N-terminal methionine, a cholix³⁸⁶ domain (SEQ ID NO: 3), and a recombinant human IL-10 (rhIL-10) domain (SEQ ID NO: 1), connected by an amino acid polypeptide spacer of glycine and serine (polyGlySer) residues (SEQ ID NO: 5). The cholix³⁸⁶ domain was a truncated form of a variant of cholix (SEQ ID NO: 2), a non-toxic mutant derived from Vibrio cholera containing 386 amino acids. The construct had a molecular weight of 125,796 Da, and an isoelectric point (pI) of 5.49.

The cholix³⁸⁶ domain facilitates active transport of the IL-10 delivery construct of SEQ ID NO: 4 across epithelial cells via vesicular transcytosis to the local gastrointestinal submucosal tissue. Targeted delivery of the rhIL-10 directly to the Lamina propria via the oral route may bypass one or more drawbacks experienced with systemic administration and translate into higher mucosal concentrations and clinically meaningful reductions in inflammation and disease.

As used in the examples herein, Drug Substance (DS) was used when referring to the lyophilized powder and Drug Product (DP) was used when referring to the tablet form.

### Example 2: Expression of the Target Construct

Plasmids containing the coding sequence (SEQ ID NO: 6) of the target construct (SEQ ID NO: 4) were constructed by cloning into the Nde I and EcoR I sites of a pET26(b) backbone. The sequence encoded by SEQ ID NO: 6 is a codon-improved sequence for expression in bacterial cells. The plasmid contained the T7 promoter and conferred kanamycin resistance. BL21 E. coli cells were transfected with the target construct plasmid using a heat shock method of transformation as follows: BL21 cells and the target construct plasmid were aliquoted into a tube and incubated on ice for 30 minutes. The tubes were then heat shocked for 8 to 12 seconds at 42° C.±2° C. in a water bath. Immediately after the heat shock, the tubes were placed in ice for 2 to 5 minutes. Media was added into each tube and the tubes were incubated for 60 minutes at 37° C. The transformed cells were plated onto LB/Kan agar plates and incubated overnight at 37° C. A single colony was then picked from the agar plate, inoculated in 50 mL of LB media, and grown overnight in a shaker flask. Glycerol stock (50%) was added to the culture, which was then filled into cryovials and stored at ≤−70° C. The pre-RCB (Research Cell Bank; the culture+glycerol stock) was then further manufactured to produce the master cell bank (MCB).

To produce the MCB, cells from the RCB were expanded in shake flasks until sufficient cell mass was accumulated, recovered by centrifugation, resuspended in cryopreservation medium, aliquoted into 300 cryovials, and cooled until frozen. The MCB was stored at −80±5° C. in a controlled access GMP facility. The MCB was manufactured and is maintained in accordance with cGMP procedures and ICH Guidelines Q5B and Q5D.

Following expansion in a shake flask, cells were transferred into a production bioreactor. Fermentation was executed in a 1500 L bioreactor in the presence of kanamycin for selective pressure. Production fermentation consists of a cell growth phase followed by an expression phase using isopropyl β-D-1-thiogalactopyranoside (IPTG) as an inducer, where the protein was expressed intracellularly as insoluble inclusion bodies. The production reactor was controlled at set pH, temperature, and dissolved oxygen level as specified in the manufacturing procedure: pH was controlled by ammonium hydroxide addition; dissolved oxygen was controlled by air and oxygen gas flows. All gases were passed through membrane filters of pore size 0.22 μm or less. The production reactor contained bacterial growth medium with defined components. Before inoculation, these ingredients were sterilized according to written standard operating procedures. The production phase was a fed batch process, where glucose-based feed media are added to maintain cell growth and culture viability.

At the end of production, the cells were harvested by centrifugation, and the cell paste processed further or frozen for processing at a later date. High-pressure homogenization was used to release the product-containing inclusion bodies. The inclusion bodies were then resuspended, washed, and separated from other cellular components by centrifugation. The inclusion bodies were either processed forward immediately or frozen for use at a later date. The cell paste and/or inclusion body slurry was stored at −20±5° C. prior to further use.

### Example 3: Refolding Optimization

Solubilization of inclusion bodies (IBs) was carried out using a high concentration (6 M) of guanidine hydrochloride, a strong chaotrope. Following solubilization, the delivery construct (without being subjected to a reducing agent or a sulfitolysis agent) was added to a refolding solution of 1 mM reduced glutathione and 0.5 mM oxidized glutathione, 1 M arginine, 250 mM sucrose, 100 mM Tris, 2 mM EDTA, pH 8.0 at 4° C. The resulting mixture was then subjected to sterile filtration (using a filter with a pore size of 0.2 μm) and tangential flow filtration (diafiltration).

### Example 4: Purification of Refolded Constructs

The refolded delivery construct from Example 3 (SEQ ID NO: 4) was subjected to anion exchange chromatography (Capto™ Q ImpRes) which inter alia, separated the active dimer species from the two major product-related impurities, residual monomer and aggregated species. As a polishing step, ceramic hydroxyapatite (CHT) chromatography was implemented as a mixed-mode orthogonal step to further reduce product and process related impurities. Gradient elutions on both chromatography steps were utilized. During elution, fractions were collected and each fraction assayed by SE-HPLC for dimer content of the target construct. Fractions containing above a specified threshold (e.g., 75%) were then pooled in order to meet the desired dimer content percentage. Following the ceramic hydroxyapatite chromatography step, the final bulk was concentrated and diafiltered using UF/DF into formulation buffer (10 mM potassium phosphate, 2% glycine, 1% sucrose, 0.3% poloxamer 188 at a pH of 7.5).

### Example 5: Lyophilization of the Liquid Intermediate

The purified liquid intermediate produced following the purification protocol described in Example 4 was transformed into a dried powder by lyophilization. Bulk lyophilization was carried out by thawing and dispensing the liquid intermediate into trays that were loaded into a lyophilizer. Control parameters during the lyophilization cycle such as temperature and vacuum pressure were executed based on time. The lyophilized powder was pooled and mixed in a low-density polyethylene (LDPE) primary liner that was placed inside a secondary LDPE liner. The second liner was heat sealed, then placed into a mylar bag, which was also heat sealed. When lyophilized, the target construct resulted in a white to off-white powder. The combined lyophilized powder is the target construct drug substance (DS).

### Example 6—Tablet Formation

Tablets containing 3 mg and 10 mg of delivery construct (SEQ ID NO: 4) were produced. Specifically, the lyophilized drug substance was blended with SMCC, crospovidone, and glyceryl dibehenate (see Table 2 below) and then compressed at a compression force of 3000 lbf to produce a 200 mg core.

TABLE 2
Composition of Tablet Core

| | Mass Per Tablet (mg) | |
|---|---|---|
| | 3 mg | 10 mg |
| Constituent | tablet | tablet |
| IL-10 Delivery Construct | 8.2 | 27.3 |
| Drug Substance | | |
| IL-10 delivery construct | 3.0 | 10.0 |
| Potassium Phosphate | 0.3 | 0.9 |
| Glycine | 3.0 | 10.0 |
| Sucrose | 1.5 | 5.0 |
| Poloxamer 188 | 0.5 | 1.5 |
| Silicified microcrystalline | 181.3 | 162.2 |
| cellulose | | |
| Crospovidone | 8.0 | 8.0 |
| Glyceryl dibehenate | 2.5 | 2.5 |

The compressed mixture was then coated with 8 mg of HPMC. Then a second coat comprising Eudragit L30D55, Eudragit FS30D, and PlasAcryl T20 was applied via spray coating to produce the final tablets. The ratio of Eudragit L30D55 to Eudragit FS30D was 30:70 (w/w) (4.1 mg Eudragit L30D55; 9.5 mg Eudragit FS30D; 1.5 mg PlasAcryl T20; solids content in final tablet).

### Example 7—Phase 2 Clinical Studies

A phase 2 clinical study was conducted of the safety and efficacy of SEQ ID NO: 4 in subjects with a prior history of ulcerative colitis (UC) who have undergone colectomy with subsequent ileal pouch-anal anastomosis (IPAA) formation and have a history of chronic antibiotic-resistant pouchitis. The study was conducted with a randomized, double-blind, multicenter design. In this study, 22 subjects were randomized in a double-blind manner to either 3 mg once daily or 10 mg once daily of an enteric-coated tablet of SEQ ID NO: 4 (as described above in Example 6) for 12 weeks in an approximately 1:1 fashion (10 individuals to the 3 mg dose and 12 individuals to the 10 mg dose). No matching placebo was used for this phase 2 study.

Specifically, 3 mg or 10 mg tablets were administered once daily by mouth in the morning approximately 30 minutes before breakfast for 12 weeks. The first dose was administered in the clinic under medical supervision, and all other doses were self-administered at home with details recorded in the subject diary.

Inclusion criteria included the following: (1) male and female subjects aged 18 to 75 years, inclusive; (2) IPAA for ulcerative colitis completed at least 1 year prior to screening; (3) active signs and symptoms of pouchitis, as follows: (a) modified Pouchitis Disease Activity Index (mPDAI) score ≥5, and (b) increased stool frequency, defined as 3 more stools per day above “Normal” (after IPAA) and an absolute total of ≥6 stools per day; (4) chronic or recurrent pouchitis, defined by: (a) ≥2 episodes within 1 year prior to or including the screening period treated with antibiotic or other prescription therapy, or, (b) maintenance antibiotic therapy taken continuously for ≥4 weeks immediately prior to the screening endoscopy; (5) antibiotic-resistant pouchitis, defined as disease remaining active despite at least 2 weeks of antibiotic therapy; (6) histologic inflammation in the pouch, defined by a Geboes score of 3.1 or greater; (7) unlikely to conceive; (8) women of childbearing potential (WOCBP) must have had a negative pregnancy test at screening and at the randomization visit prior to the first dose of study drug; (9) were able to participate fully in all aspects of this clinical trial; and (10) written informed consent was obtained and fully documented.

For criterion (3), “increased stool frequency” was determined based on the difference between “Normal” and screening stool frequency. “Normal” was the stool frequency achieved post-IPAA when the subject's bowel function was most settled. This typically occurs approximately 1 year after IPAA and was supported by documentation in the subject's medical records. To enter the study, subjects must have experienced 6 or more stools per day, and this value must be 3 or more stools per day greater than the “Normal” value, as defined above. Stool frequency was recorded daily in the subject diary, and the subject was instructed that a stool is defined as a trip to the toilet when the subject has either a bowel movement, or passes blood alone, blood and mucus, or mucus only.

Exclusion criteria included the following: (1) known Crohn's disease (CD) or suspected CD of the pouch, defined as complex perianal/pouch fistula and/or extensive length of pre-pouch ileitis with deep ulceration; (2) diagnosed or suspected irritable pouch syndrome (IPS); (3) isolated or predominant cuffitis; (4) mechanical complications of the pouch such as stricture or fistula(e) that preclude evaluation of the pouch and terminal ileum; (5) fecal incontinence due to anal sphincter dysfunction; (6) pelvic sepsis within 12 months prior to screening; (7) planned surgery for UC, or any other elective surgery within the time frame of the study, (8) diverting stoma; (9) current bacterial or parasitic pathogenic enteric infection; (10) no more than 60% of enrolled subjects may have prior failure of any biologics for pouchitis; and (11) subjects who have used prior biologic therapies must have discontinued use within 12 weeks or 5 half-lives of screening (or within 4 weeks if drug levels are undetectable).

Data from the subjects were collected before the first administration of the drug, the day of randomization, during the course of the study, at the end of the treatment period (12 weeks), and during subsequent safety follow up (4 weeks post treatment). Specifically, data were gathered for the following efficacy endpoints: (1) proportion of subjects with a stool frequency response at Week 12; defined as (a) a reduction of ≥3 stools and ≥30% reduction in number of stools from a baseline (i.e., average daily stools for the 7-day period prior to randomization and administration of the first dose), OR (b) back to (or below) postoperative Normal number of stools (often number of stools approximately 1 year after IPAA); (2) proportion of subjects with histologic healing at Week 12, defined as neutrophil infiltration of <5% of crypts, no crypt destruction, and no erosions, ulcerations, or granulation tissue (Geboes score ≤3.1); and (3) proportion of subjects with histologic response at Week 12; defined as a reduction in Pouchitis Disease Activity Index (PDAI) histology subscore ≥2 points from baseline (i.e., prior to administration of the first dose) or a PDAI histology subscore of 0; (4) proportion of subjects with ≥50% reduction in Simple Endoscopic Score for Crohn's Disease (SES-CD) score at Week 12 (area within 1 cm of the pouch suture line was not included in the endoscopic evaluation); and (5) proportion of subjects with minimal histologic activity at Week 12; defined as PDAI neutrophil score ≤1 and ulcer score of 0.

### Geboes Scoring System

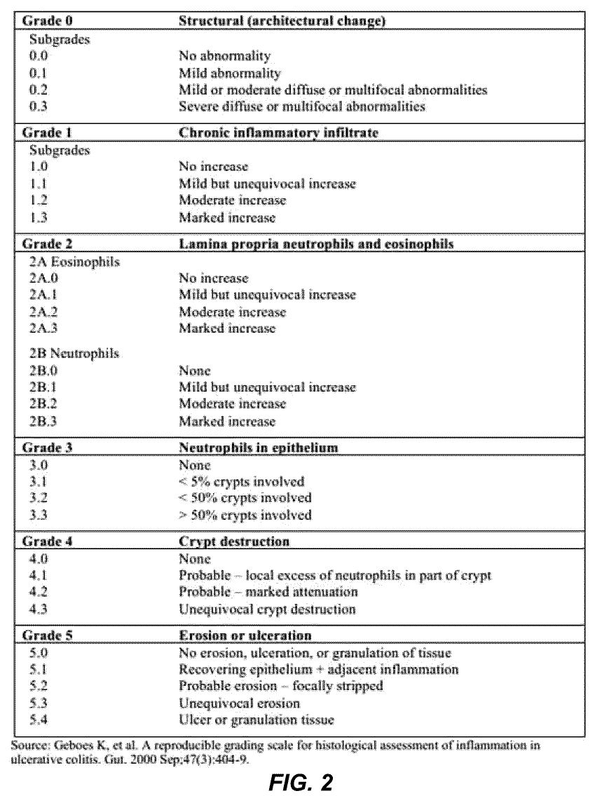

The Geboes scoring system is a stepwise ordinal grading system for histological assessment of disease severity in UC. Geboes K, Riddell R, Ost A, Jensfelt B, Persson T, Lofberg R. A reproducible grading scale for histological assessment of inflammation in ulcerative colitis. Gut. 2000 September; 47(3):404-9. The scoring system progressively grades disease severity by assessing histological items and grading histological change as grade 0 (structural change only), 1 (chronic cell infiltrations), 2 A (Lamina propria neutrophils), 2B (Lamina propria eosinophils), 3 (neutrophils in the epithelium), 4 (crypt destruction), and 5 (erosion or ulceration). Each of the grades is subdivided into subgrades, based upon the severity of tissue abnormalities or the extent of cell infiltration (FIG. 2). Subgrades are assessed from the worst area of the biopsy. Higher Geboes scores are indicative of more severe disease activity.

### Pouchitis Disease Activity Index (PDAI) and Modified PDAI

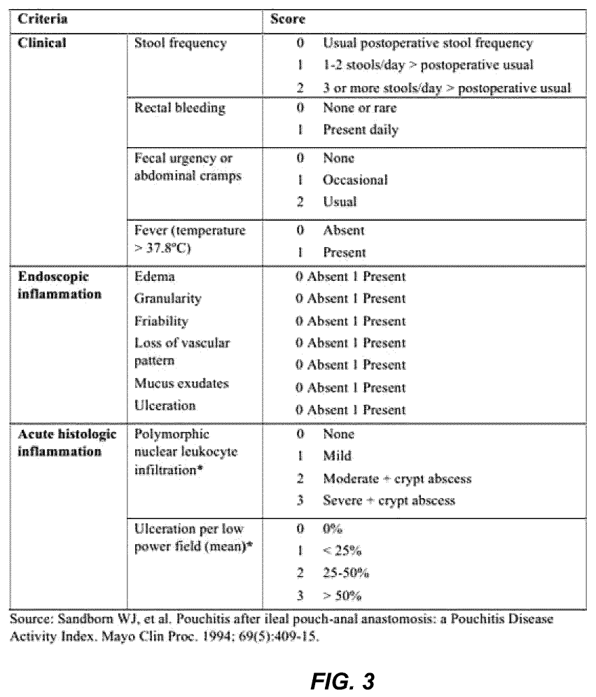

The PDAI was developed as simple, objective, quantitative criteria for assessing pouch inflammation after IPAA. Sandborn W J, Tremaine W J, Batts K P, Pemberton J H, Phillips S F. Pouchitis after ileal pouch-anal anastomosis: a Pouchitis Disease Activity Index, Mayo Clin Proc. 1994 69(5):409-15. The total PDAI score (range, 0-18) (see FIG. 3) is calculated by adding up the scores for (a) clinical symptoms, (b) endoscopic findings and (c) histologic findings, with higher scores indicating more severe disease activity:
((a) clinical symptoms (score range 0-6) which include:
   (i) stool frequency:
      (score 0) usual postoperative stool frequency,
      (score 1) 1-2 stools/day>postoperative usual,
      (score 2) 3 or more stools/day>postoperative usual;
   (ii) rectal bleeding:
      (score 0) none or rare,
      (score 1) present daily;
   (iii) fecal urgency or abdominal cramps:
      (score 0) none,
      (score 1) occasional,
      (score 2) usual;
   (iv) fever (temperature >37.8° C.):
      (score 0) absent,
      (score 1) present;
(b) endoscopic findings (score range 0-6) which include:
   (i) Edema: (score 0) absent, (score 1) present;
   (ii) Granularity: (score 0) absent, (score 1) present;
   (iii) Friability: (score 0) absent, (score 1) present;
   (iv) Less of vascular pattern: (score 0) absent, (score 1) present;
   (v) Mucus exudates: (score 0) absent, (score 1) present;
   (vi) Ulceration: (score 0) absent, (score 1) present; and
(c) histologic findings (score range 0-6) which include:
   (i) polymorphic nuclear leukocyte infiltration:
      (score 0) none,
      (score 1) mild,
      (score 2) moderate+crypt abcess
      (score 3) severe+crypt abcess;
(ii) ulceration per low power field (mean):
   (score 0) 0%,
   (score 1) <25%,
   (score 2) 25-50%,
   (score 3) >50%).

In some embodiments, baseline pouchitis is indicated by a score of greater than or equal to 7 points (Total PDAI Score: 0-18) (see Gionchetti P, Amadini C, Rizzello F, Venturi A, Poggioli G, Campieri M. Diagnosis and treatment of pouchitis. Best Pract Res Clin Gastroenterol. 2003 February; 17(1):75-87). In some embodiments, a treatment response is indicated by a greater than or equal to 3 point reduction from baseline, and in some embodiments remission is indicated by a score of 0 (see Ma C, Athayde J, Davies S C, Parker C E, Nguyen™, Khanna R, et al. Identifying outcomes in clinical trials of pouchitis for the development of a core outcome set. Clin Gastroenterol Hepatol. 2019 July; 17(8):1637-40). The mPDAI consists only of the clinical symptoms and endoscopic findings. Shen B, Achkar J P, Connor J T, Ormsby A H, Remzi F H, Bevins C L, et al. Modified pouchitis disease activity index: a simplified approach to the diagnosis of pouchitis. Diseases of the Colon & Rectum. 2003 46(6):748-53. A cutoff of 5 differentiates patients with pouchitis (mPDAI≥5) from patients without pouchitis (mPDAI<5).

### Simple Endoscopic Score for Crohn's Disease (SES-CD)

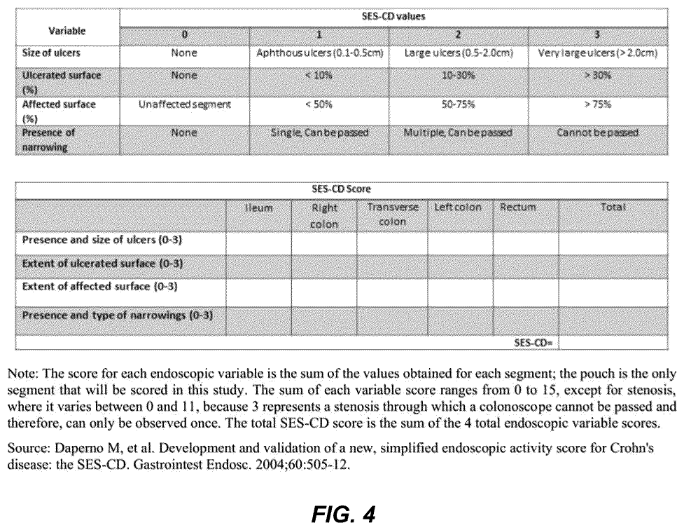

The SES-CD scores four endoscopic variables including ulcer size, proportion of ulcerated surface, proportion of the surface area affected by any disease lesion, and stenosis. Daperno M, D'Haens G, Van Assche G, Baert F, Bulois P, Maunoury V, et al. Development and validation of a new, simplified endoscopic activity score for Crohn's disease: the SES-CD, Gastrointest Endosc. 2004 October; 60(4):505-12. Each variable is scored from 0 to 3, with higher scores representing more severe disease. In subjects with Crohn's Disease, the SES-CD is scored in each of 5 intestinal segments, but for this study of subjects with pouchitis, only 1 segment (the pouch) was scored. Therefore, the SES-CD scores in this study could range from 0 to a maximum of 12 points, with higher scores representing more severe disease (FIG. 4).

St. Mark's Incontinence/Urgency Scale

The St. Mark's Incontinence/Urgency Scale combines components of 3 predicate scales (Pescatori, Wexner, AMD scales) (see Vaizey C J, Carapeti E, Cahill J A, Kamm M A. Prospective comparison of faecal incontinence grading systems. Gut. 1999 January; 44(1):77-80). The scale includes elements to assess fecal urgency and use of antidiarrhea drugs as well as alterations in lifestyle. Four items for incontinence and lifestyle impact are assessed with a 5-point frequency scale and 3 items are assessed with a yes/no response to provide a total score. Higher scores indicate greater incontinence and lifestyle impacts.

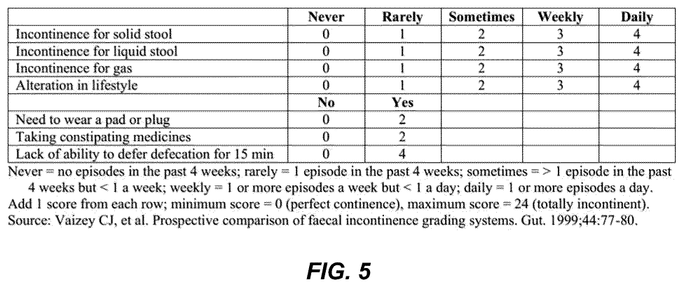

St. Mark's Scale of Fecal Incontinence and Urgency is shown in FIG. 5: Incontinence for solid stool: (0) never, (1) rarely, (2) sometimes, (3) weekly, (4) daily; Incontinence for liquid stool: (0) never, (1) rarely, (2) sometimes, (3) weekly, (4) daily; Incontinence for gas: (0) never, (1) rarely, (2) sometimes, (3) weekly, (4) daily; Alteration in lifestyle: (0) never, (1) rarely, (2) sometimes, (3) weekly, (4) daily; Need to wear a pad or plug: (0) no, (2) yes; Taking constipating medicines: (0) no, (2) yes; Lack of ability to defer defecation for 15 min: (0) no; (4) yes.

For reference, never=no episodes in the past 4 weeks; rarely=1 episode in the past 4 weeks; sometimes=>1 episode in the past 4 weeks but <1 a week; weekly=1 or more episodes a week but <1 a day; daily=1 or more episodes a day.

To arrive at the St. Marks score, add 1 score from each row/category, wherein a minimum score=0 (perfect continence) and a maximum score=24 (totally incontinent).

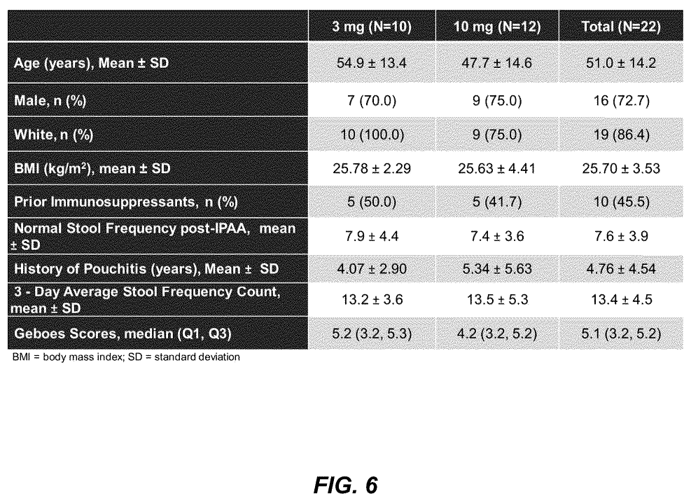

The baseline characteristics of the patients in the phase 2 clinical study are shown in FIG. 6.

### Analysis of Immune Cell Population by Immunohistochemistry

Whole tissue biopsies were obtained from each subject. Tissue sections were stained and imaged using antibodies to FOXP3 and CD163. Images underwent digital imaging analysis to identify the Lamina propria tissue compartment and determine absolute number of total cells and IHC marker positive cells in this compartment. Results were expressed as proportion of IHC marker positive cells out of the total number of cells detected, per subject per visit.

### Analysis of Phospho-STAT3 to Total STAT3 Ratio Via Enzyme-Linked Immunosorbent Assay (ELISA)

The MesoScale Discovery (MSD) platform and Phospho-STAT3 (Tyr705) [cat. #K150SVD-1] kit was used to measure the levels of total-STAT3 and phosphorylated-STAT3 in colonic biopsies. Background subtracted and standard quantitated data was obtained. Assay output values were further normalized to the ratio of p-STAT3 to total STAT3 per subject per visit.

### Analysis of Microbiome Diversity

Fecal microbiome samples were obtained from each subject, both at randomization (i.e., before treatment) and at the conclusion of the study (after 12 weeks of treatment). DNA was extracted using an automated platform (ZymoBIOMICS®-96 MagBead DNA Kit). DNA samples were then profiled with shotgun metagenomic sequencing. Sequencing libraries were prepared with either the KAPA™ HyperPlus Library Preparation Kit (Kapa Biosystems, Wilmington, MA) with up to 100 ng DNA input following the manufacturer's protocol using internal single-index 8 bp barcodes with TruSeq® adapters (Illumina, San Diego, CA) or the Nextera® DNA Flex Library Prep Kit (Illumina, San Diego, CA) with up to 100 ng DNA input following the manufacturers protocol using internal dual-index 8 bp barcodes with Nextera® adapters (Illumina, San Diego, CA). All libraries were quantified with TapeStation® (Agilent Technologies, Santa Clara, CA) and then pooled in equal abundance. The final pool was quantified using qPCR. The final library was sequenced on either the Illumina HiSeq® or the Illumina NovaSeq® platforms.

Raw sequence reads were trimmed to remove low quality fractions and adapters with Trimmomatic-0.33 (Bolger et al., 2014): quality trimming by sliding window with 6 bp window size and a quality cutoff of 20. Reads with size lower than 70 bp were removed. Antimicrobial resistance and virulence factor gene identification was performed with the DIAMOND sequence aligner (Buchfink et al., 2015). Microbial composition was profiled with Centrifuge (Kim et al., 2016) using bacterial, viral, fungal, mouse, and human genome datasets. Strain-level abundance information was extracted from the Centrifuge outputs and further analyzed: (1) to perform alpha- and beta-diversity analyses; (2) to create microbial composition barplots with QIIME (Caporaso et al., 2012); (3) to create taxa abundance heatmaps with hierarchical clustering (based on Bray-Curtis dissimilarity); and (4) for biomarker discovery with LEfSe (Segata et al., 2011) with default settings (p>0.05 and LDA effect size >2).

### Results

Both the 3 mg and 10 mg doses were safe and well tolerated. As shown in Table 3, more than 30% of the individuals in both arms of the study exhibited a stool frequency response at Week 12, defined as (a) a reduction of ≥3 stools and ≥30% reduction in number of stools from a baseline (i.e., average daily stools for the 7-day period prior to randomization and administration of the first dose) or (b) back to (or below) postoperative Normal number of stools (often number of stools approximately 1 year after IPAA).

TABLE 3

| Stool Frequency Response (%) at Week 12 | |
|---|---|
| | Responders |
| 3 mg dose of SEQ ID NO: 4 | 40% (4/10) |
| 10 mg dose of SEQ ID NO: 4 | 33.3% (4/12) |
| Total | 36.4% (8/22) |

As shown in Table 4, a rapid onset of stool frequency response was observed as early as Week 2 and clinical response was maintained through duration of treatment in both dose arms.

TABLE 4
Stool Frequency Response (%) by Visit

| | Responders | Responders | Responders | Responders | Responders |
|---|---|---|---|---|---|
| | at Week 2 | at Week 6 | at Week 8 | at Week 10 | at Week 12 |
| | (%) | (%) | (%) | (%) | (%) |
| 3 mg dose of | 30.0 | 40.0 | 40.0 | 30.0 | 40.0 |
| SEQ ID NO: 4 | | | | | |
| 10 mg dose of | 16.7 | 41.7 | 33.3 | 41.7 | 33.3 |
| SEQ ID NO: 4 | | | | | |
| Total | 22.7 | 40.9 | 36.4 | 36.4 | 36.4 |

As shown in Table 5, a subset of the individuals in both dose arms of the study exhibited a histologic healing response at Week 12, defined as neutrophil infiltration of <5% of crypts, no crypt destruction, and no erosions, ulcerations, or granulation tissue (Geboes score ≤3.1).

TABLE 5

| Histologic Healing Response (%) at Week 12 | |
|---|---|
| | Responders |
| 3 mg dose of SEQ ID NO: 4 | 20.0% (2/10) |
| 10 mg dose of SEQ ID NO: 4 | 25.0% (3/12) |
| Total | 22.7% (5/22) |

As shown in Table 6, a subset of the individuals in both dose arms of the study exhibited a histologic response at Week 12, defined as a reduction in Pouchitis Disease Activity Index (PDAI) histology subscore ≥2 points from baseline (i.e., prior to administration of the first dose) or a PDAI histology subscore of 0.

TABLE 6

| Histologic Response (%) at Week 12 | |
|---|---|
| | Responders |
| 3 mg dose of SEQ ID NO: 4 | 20.0% (2/10) |
| 10 mg dose of SEQ ID NO: 4 | 16.7% (2/12) |
| Total | 18.2% (4/22) |

As shown in Table 7, a subset of the individuals in both dose arms of the study exhibited a minimal histologic activity response at Week 12, defined as a PDAI neutrophil score ≤1 and ulcer score of 0.

TABLE 7

| Minimal Histologic Activity Response (%) at Week 12 | |
|---|---|
| | Responders |
| 3 mg dose of SEQ ID NO: 4 | 20.0% (2/10) |
| 10 mg dose of SEQ ID NO: 4 | 33.3% (4/12) |
| Total | 27.3% (6/22) |

As shown in Table 8, a subset of the individuals in both dose arms of the study exhibited an SES-CD response at Week 12, defined as a ≥50% reduction in Simple Endoscopic Score for Crohn's Disease (SES-CD) score at Week 12 (area within 1 cm of the pouch suture line was not included in the endoscopic evaluation).

TABLE 8

| SES-CD Response (%) at Week 12 | |
|---|---|
| | Responders |
| 3 mg dose of SEQ ID NO: 4 | 10.0% (1/10) |
| 10 mg dose of SEQ ID NO: 4 | 8.3% (1/12) |
| Total | 9.1% (2/22) |

As shown in Table 9, individuals showed a reduction in mean percent change from baseline in Fecal Urgency or Abdominal Cramp subscore via PDAI over 12 weeks. Individuals reported symptoms daily in an electronic diary.

TABLE 9
Mean Percent Change From Baseline in PDAI
Fecal Urgency or Abdominal Cramp Subscore

| | Week 2 | Week 6 | Week 8 | Week 10 | Week 12 |
|---|---|---|---|---|---|
| | (%) | (%) | (%) | (%) | (%) |
| 3 mg dose of | −22.2 | −33.3 | −14.3 | −60.0 | −71.4 |
| SEQ ID NO: 4 | | | | | |
| 10 mg dose of | −16.7 | −36.4 | −50.0 | −40.0 | −44.4 |
| SEQ ID NO: 4 | | | | | |
| Total | −19.0 | −35.0 | −35.3 | −46.7 | −56.3 |

As shown in Table 10, individuals showed an improvement in the mean percentage change from baseline in incontinence and urgency score via St. Mark's Scores at Week 12.

TABLE 10
Mean % Change from Baseline In
Incontinence and Urgency at Week 12

| | Mean % Change from |
|---|---|
| | Baseline at Week 12 |
| 3 mg dose of SEQ ID NO: 4 | −17.7% |
| 10 mg dose of SEQ ID NO: 4 | −13.8% |

A shown in Table 11, a subset of individuals in both dose arms of the study exhibited partial mPDAI responses at Week 12, defined as a reduction in mPDAI score greater than or equal to 2 points from baseline.

TABLE 11

| Partial mPDAI Response (%) at Week 12 | |
|---|---|
| | Responders |
| 3 mg dose of SEQ ID NO: 4 | 20.0% (2/10) |
| 10 mg dose of SEQ ID NO: 4 | 33.3% (4/12) |
| Total | 27.3% (6/22) |

As shown in Table 12, a subset of individuals in both dose arms of the study exhibited PDAI improvement at Week 12, defined as a PDAI score of less than 7 points and reduction of overall score greater than or equal to 3 points from baseline.

TABLE 12

| PDAI Improvement (%) at Week 12 | |
|---|---|
| | Responders |
| 3 mg dose of SEQ ID NO: 4 | 10.0% (1/10) |
| 10 mg dose of SEQ ID NO: 4 | 16.7% (2/12) |
| Total | 13.6% (3/22) |

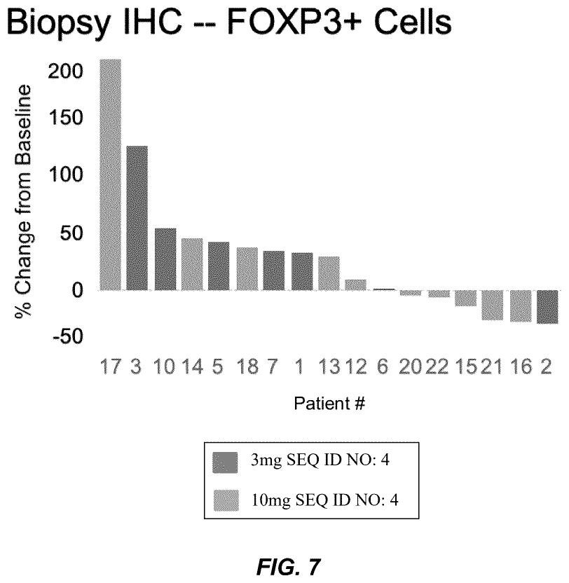

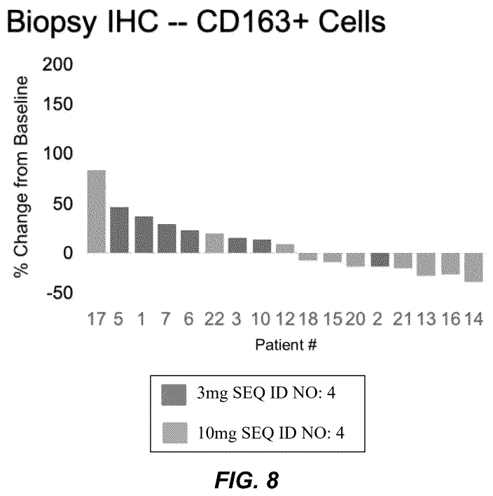

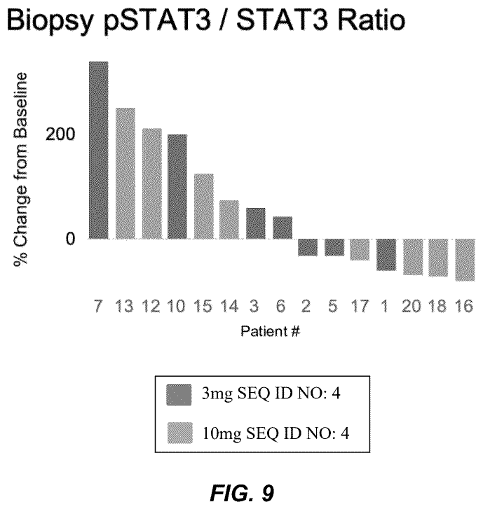

Quantitation of immune cell markers was performed by immunohistochemistry of mucosal biopsies as described above. FOXP3 is a biomarker for T-regulatory cells, and CD163 is a biomarker for M2-macrophages. As shown in FIG. 7, a trend of increased FOXP3+ T-regulatory cells in the Lamina propria was observed at the end of treatment in both treatment groups relative to baseline levels prior to treatment. Similarly, as shown in FIG. 8, a trend of increased CD163+M2-macrophages in the Lamina propria was observed in both treatment groups relative to baseline levels prior to treatment. In addition, a trend of increase in the activation state of STAT3 as determined by ELISA (see description above), presented as the level of p-STAT3/STAT3, was observed at the end of the treatment (FIG. 9). Collectively, the results are consistent with a net positive immune cell response to the IL-10 delivery construct in patients treated with the enteric-coated tablet of SEQ ID NO: 4.

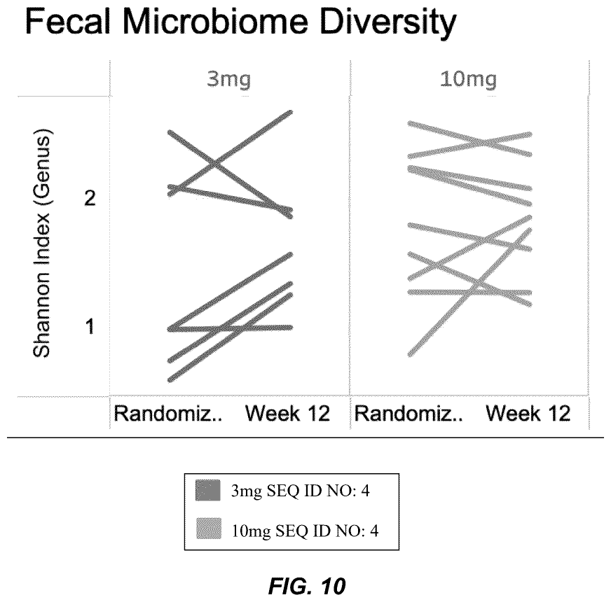

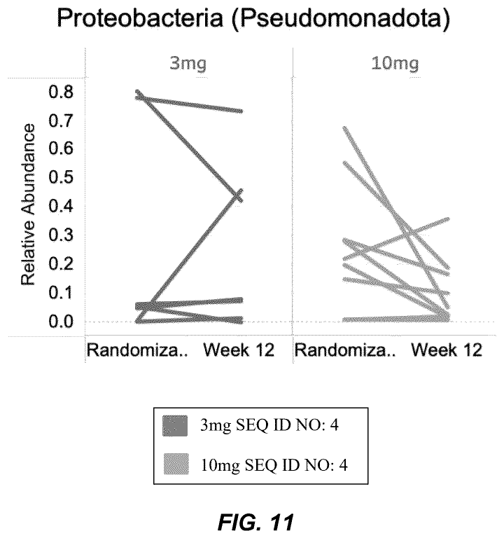

Diversity of the fecal microbiome samples from the patients were analyzed by shotgun metagenomics as described above. As shown in FIG. 10, the Alpha Shannon diversity was increased in four out of seven patients treated with 3 mg tablets and in three out of nine patients treated with 10 mg tablets. The relative abundance of Proteobacteria [Pseudomonadota], shown in FIG. 11, was decreased in three out of seven patients treated with 3 mg tablets and in six out of nine patients treated with 10 mg tablet.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-44. (canceled)

45. A method of treating an inflammatory disease of the gastrointestinal tract in a human subject to decrease stool frequency, the method comprising:
orally administering a therapeutically effective dose of a delivery construct consisting of SEQ ID NO: 4 to the human subject,
wherein the oral administration of the therapeutically effective dose results in a decrease in stool frequency of the human subject, wherein the inflammatory disease of the intestinal tract is pouchitis, and wherein the decrease in stool frequency:
(a) is (i) greater than or equal to three stools per day relative to stool frequency of the human subject prior to administration of the delivery construct to the human subject and (ii) greater than or equal to 30% relative to stool frequency of the human subject prior to administration of the delivery construct to the human subject;
(b) results in a stool frequency that is at or below the stool frequency of the human subject when the human subject's bowel function was most settled after ileal pouch anal anastomosis; or
(c) results in a stool frequency that is at or below the stool frequency of the human subject one year after ileal pouch anal anastomosis.

46. The method of claim 45, wherein the inflammatory disease of the intestinal tract is pouchitis.

47. The method of claim 45, wherein the therapeutically effective dose of the delivery construct is 3 mg of the delivery construct.

48. The method of claim 45, wherein the therapeutically effective dose of the delivery construct is 10 mg of the delivery construct.

49. The method of claim 45, wherein the therapeutically effective dose of the delivery construct is administered daily.

50. The method of claim 45, wherein the therapeutically effective dose of the delivery construct is administered to the subject over a period of at least 12 weeks.

51. The method of claim 45, wherein the oral administration of the therapeutically effective dose results in a histologic response, wherein the histologic response comprises neutrophil infiltration in less than 5% of crypts, no erosions, no ulcerations, no granulation, or a Geboes score of less than or equal to 3.1.

52. The method of claim 51, wherein the histologic response is determined in week 12 after initial administration of the delivery construct to the human subject.

53. The method of claim 45, wherein the oral administration of the therapeutically effective dose results in increased FOXP3+ T-regulatory cells in the Lamina propria.

54. The method of claim 45, wherein the oral administration of the therapeutically effective dose results in increased CD163+/M2-macrophages in the Lamina propria.

55. The method of claim 45, wherein the oral administration of the therapeutically effective dose results in an increased phospho-STAT3 (Tyr705) to total STAT3 ratio in colon.

56. The method of claim 45, wherein the oral administration of the therapeutically effective dose results in increased microbiome diversity in feces (e.g., as determined by a Shannon diversity score).

57. The method of claim 45, wherein the oral administration of the therapeutically effective dose results in decreased relative abundance of Proteobacteria in feces.

58. The method of claim 45, wherein the inflammatory disease of the intestinal tract is chronic antibiotic-resistant pouchitis, chronic antibiotic-refractory pouchitis, or chronic antibiotic-dependent pouchitis.

59. The method of claim 45, wherein the human subject has failed at least one round of antibiotic therapy prior to oral administration of the therapeutically effective dose of the delivery construct to the human subject.

60. The method of claim 45, wherein the human subject does not receive an antibiotic during a course of treatment that comprises orally administering the therapeutically effective dose of the delivery construct consisting of SEQ ID NO: 4 to the human subject.

61. The method of claim 45, wherein the delivery construct is orally administered via a tablet.

62. The method of claim 61, wherein the tablet is an enteric-coated tablet.

63. The method of claim 45, wherein the human subject has been identified as having pouchitis prior to oral administration of the therapeutically effective dose.

64. An oral formulation comprising a therapeutically effective dose of a delivery construct consisting of SEQ ID NO: 4 and an enteric coating.
